(19) United States
(12) Patent Application Publication
(10) Pub. No.: US 2013/0310715 A1
(43) Pub. Date: Nov. 21, 2013

(54) APPARATUS FOR THE TREATMENT OF HYPERHIDROSIS

(71) Applicant: Afschin Fatemi, Dusseldorf (DE)

(72) Inventor: Afschin Fatemi, Dusseldorf (DE)

(21) Appl. No.: 13/990,287

(22) PCT Filed: Nov. 30, 2011
(86) PCT No.: PCT/EP2011/006001

§ 371 Date: Jun. 10, 2013

(30) Foreign Application Priority Data
Nov. 30, 2010 (EP) 10015131.5

(51) Int. Cl. A61N 5/00; A61N 7/02

(52) U.S. Cl. CPC A61N 5/00; A61N 7/02
USPC 601/3; 607/101

(57) ABSTRACT

Provided herein are the novel uses of apparatuses delivering electromagnetic energy through an external skin surface to an underlying sweat glands containing tissue site for the treatment of hyperhidrosis. Electromagnetic energy delivery means are provided and include an interface surface configured to conform to the exterior skin layer surface. Electrode means are coupled to the electromagnetic energy delivery means and configured to transfer electromagnetic energy through the interface surface and the skin layer surface to the underlying sweat glands containing tissue site.

### FIELD OF THE DISCLOSURE

The technology provided herein relates to medical systems, methods novel use of electromagnetic energy for to the treatment of hyperhidrosis. In particular, the present application relates to methods and apparatuses for reducing sweat production via the removal, disablement, incapacitation of apocrine and eccrine glands in the dermal and subcutaneous tissue.

### BACKGROUND

Focal hyperhidrosis is an entity, in which patients sweat excessively in selected areas of the body, most commonly axillae, palms or feet. Patients sweat in general more than 30 mg/min, independent of external or internal temperatures.

Focal hyperhidrosis can be followed by other diseases such as intertrigo, mycosis, bacterial infection, erythema, pigmentation, bromhidrosis, general infection or cold.

Treatment options include topical aluminum chloride or iontopheresis, which are both not very effective and are accompanied by discomfort. Same as with not so effective systemic medications, there is an injections of botulinum toxin are effective, but the effect lasts only up to six months.

Permanent options are surgical. Although effective, even the lesser invasive operations have possible surgical risks and downtime.

It is known that energy-based therapies like laser therapy can be applied to tissue throughout the body to achieve numerous therapeutic and/or aesthetic results.

However, there is no effective nonsurgical, low-risk treatment of focal hyperhidrosis so far. There exists a need for the treatment of hyperhidrosis without surgical scarring or side effects of more invasive techniques.

Therefore, effective and improved methods, systems and apparatuses that are able to treat hyperhidrosis non-invasively are needed.

### SUMMARY OF THE DISCLOSURE

In a first aspect, embodiments of this disclosure provide the use of electromagnetic energy in the treatment of hyperhidrosis.

In a further aspect, embodiments of this disclosure relate to methods of treating and preventing focal hyperhidrosis which comprise administering to a patient in need of such treatment or prevention electromagnetic energy.

In still another aspect, embodiments of this disclosure relate to a medical system for the treatment of hyperhidrosis.

In a further aspect, the present disclosure pertains to methods of non-invasively applying ultrasound waves, preferably high intense focused ultrasound to reduce the severity of the symptoms of focal hyperhidrosis. The method can reduce the severity of excessive sweating or other fluid weeping from the skin, erythematous, leathery appearance and texture, and associated pain and discomfort.

In a further aspect, the present disclosure pertains to methods of nonablative applying capacitive radiofrequency energy.

In one aspect, the methods involve non-invasively treating the affected area with one of the systems of the present disclosure, a gel sheet and a transducer, generally penetrating the skin deeply and creating a lesion in the target area.

### BRIEF DESCRIPTION OF THE FIGURES

FIG. 1 is a perspective view of an example of an apparatus for applying electromagnetic energy through the skin in order to cause a reduction of sweat glands.

FIG. 2 shows a schematic treatment plan for hyperhidrosis using high intense focused ultrasound.

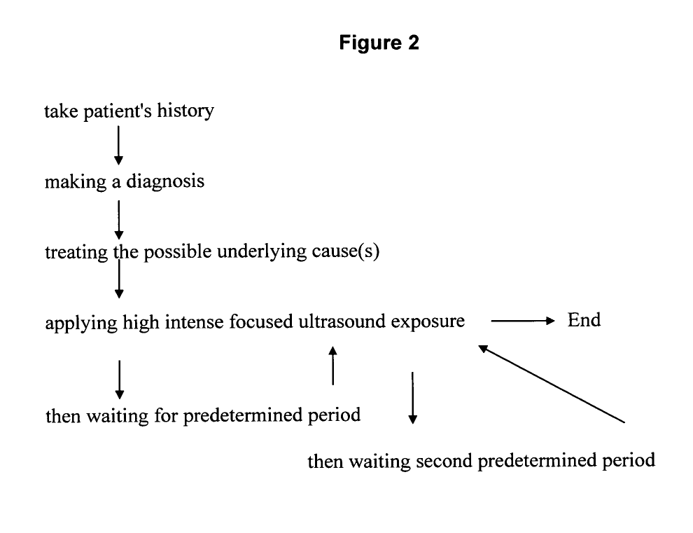

FIG. 3 shows a schematic treatment plan for hyperhidrosis using nonablative radiofrequency.

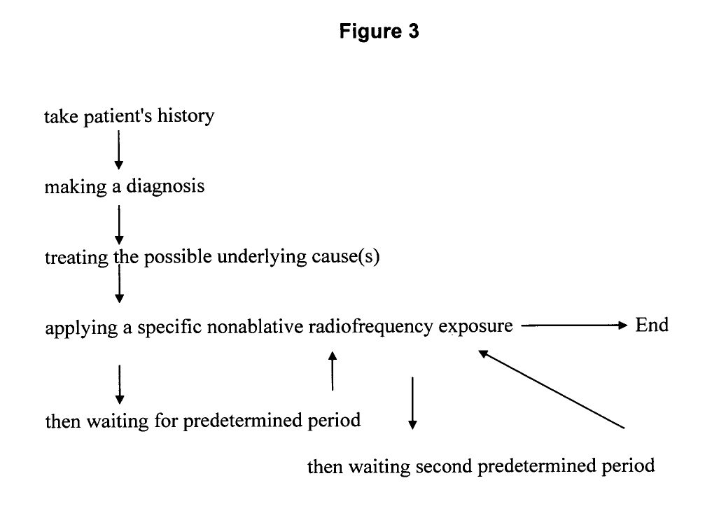

### DETAILED DESCRIPTION OF THIS DISCLOSURE

Disclosed herein are medical systems, novel uses and methods of non-invasively applying electromagnetic energy for the treatment of hyperhidrosis.

The present disclosure provides a method for reducing sweat glands e.g. for the treatment of hyperhidrosis below a tissue surface in a selected tissue site. The reduction or elimination of sweat glands can be done preferably transcutaneously, with a uniform reverse thermal gradient, percutaneously, transmucosally, permucosally, or through a device including but not limited to an endoscope. An electromagnetic energy apparatus is provided and includes an electromagnetic energy source and a delivery device. The delivery device is positioned on the tissue surface. Electromagnetic energy is produced from the electromagnetic energy source and delivered through the tissue surface to the selected tissue site for a sufficient time to reduce sweat glands in the selected tissue site. No deeper than a second degree burn is formed on the tissue surface. This method is particularly useful in soft tissue sites and for the treatment of hyperhidrosis.

However, to be able to treat hyperhidrosis effectively, it is crucial to destroy most of the sweat glands, not just a few of them. With the methods according to the present disclosure it is possible to reduce a plurality of sweat glands in the selected sweat glands containing tissue site.

Hyperhidrosis can either be generalized or localized to specific parts of the body. Hands, feet, armpits, and the groin area are among the most active regions of perspiration due to the relatively high concentration of sweat glands; however, any part of the body may be affected. Hyperhidrosis can also be classified depending by onset, congenital or acquired. Primary hyperhidrosis is found to start during adolescence or even before and seems to be inherited as an autosomal dominant genetic trait. Primary hyperhidrosis must be distinguished from secondary hyperhidrosis, which can start at any point in life. The latter form may be due to a disorder of the thyroid or pituitary gland, diabetes mellitus, tumors, gout, menopause, certain drugs, or mercury poisoning.

Hyperhidrosis may be also divided into palmoplantar (symptomatic sweating of primarily the hands or feet), gustatory and generalized hyperhidrosis. Alternatively, hyperhidrosis may be classified according to the amount of skin that is affected and its possible causes.

The methods of the present disclosure use an electromagnetic energy source to apply electromagnetic energy to a selected tissue site. The electromagnetic energy can be delivered transcutaneously, with a reverse thermal gradient, percutaneously, transmucosally, permucosally, or through a device including but not limited to an endoscope.

Additionally, the methods of the present invention provide for the reduction of sweat glands underlying a tissue surface area. The overlying layer of tissue is not ablated. No deeper than a second degree burn is produced in the overlying layer of tissue, and preferably no deeper than a first degree burn.

Suitable applications for the methods of the present disclosure include but are not limited to the treatment of hyperhidrosis, bromhidrosis, hidradenitis suppurativa, hypohidrotic ectodermal dysplasia, hypotrichosis, anodontia, hypohidrosis, hidrotic ectodermal dysplasia, uremic frost, neoplasms, syringomata, and various forms of miliarias. Preferably, the methods according to the present disclosure are useful for the treatment of hyperhidrosis, in particulat for the treatment of Hyperhidrosis axillaris and Hyperhidrosis palmaris.

In one embodiment, an apparatus is used to create the uniform reverse thermal gradient is a composite heating pad that has both cooling elements and electromagnetic delivery devices. The heating pad is configured to the topography of the treatment area and is incorporated into an elastic garment. Partial reduction of sweat glands is achieved with each treatment. Thermal transducers measure the surface temperature of the treatment area to avoid blistering. In one embodiment the deeper dermis is heated to above 65 degrees for sweat glands destruction. Sequential treatments are designed to allow for more precision of the end result. Areas of application are not confined by requirements to either hide surgical incisions or transition along aesthetic boundaries.

The radiofrequency treatment according to the present disclosure delivers almost uniform and deep heating since only with almost uniform and deep heating it is possible to coagulate enough sweat glands to treat hyperhidrosis effectively.

A uniform reverse thermal gradient means that the energy delivery surface uniformly distributes the thermal gradient through the skin epidermis surface to the selected sweat glands containing tissue site.

Various types of electromagnetic energy can be utilized with the present disclosure. Electromagnetic energy may be any kind that can cause cell heating or physical destruction by being applied to collagen tissue. However, in advantageous embodiments of the present disclosure the electromagnetic energy sources is RF or ultrasound, preferably a high intensity focused ultrasound (HIFU).

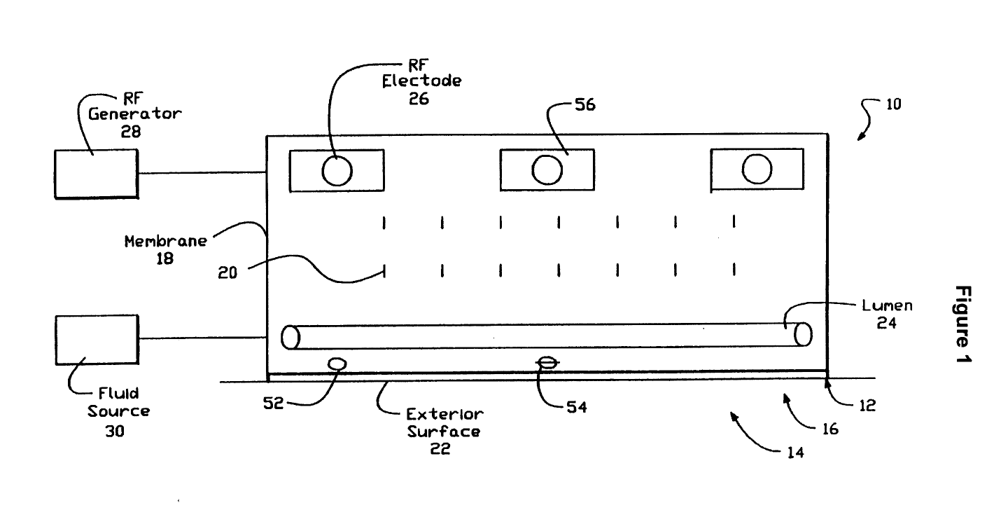

FIG. 1 shows an example of an apparatus 10 applies electromagnetic energy through a skin layer 12, such as the epidermis, and to the underlying sweat glands tissue 14 without substantially modifying melanocytes and other epithelial cells 16 found in the lower layer of epidermis layer 12. A porous membrane 18 is adapted to receive an electrolytic solution 20. Porous membrane 18 becomes inflated to substantially conform a contacting exterior surface 22 of porous membrane 18 which is in close thermal contact with epidermis 12. Porous membrane 18 includes a cooling lumen 24 for receiving a cooling fluid that imparts a cooling effect on epidermis layer 12.

One or more electromagnetic electrodes 26 are positioned at various places in porous membrane 18. In one embodiment, electromagnetic electrodes 26 are positioned on a side that is substantially opposing to contacting exterior surface 22. In other embodiments, electromagnetic electrodes 26 are placed closer to cooling lumen 24. In embodiment particularly suitable for the hips, porous membrane is about 20 cm by 30 cm, with an oval shape.

An electromagnetic power source 28 is coupled to electromagnetic electrodes 26 and a source of electrolytic solution 30 is coupled to porous membrane 18.

Various examples of an apparatus useful for the methods according to the present disclosure are described in the U.S. Pat. No. 6,241,753, U.S. Pat. No. 6,311,090, U.S. Pat. No. 6,350,276, U.S. Pat. No. 6,377,855, U.S. Pat. No. 6,381,497, U.S. Pat. No. 6,381,498, U.S. Pat. No. 6,387,380, U.S. Pat. No. 6,425,912, U.S. Pat. No. 6,405,090, U.S. Pat. No. 6,453,202, U.S. Pat. No. 7,022,121, U.S. Pat. No. 7,258,674 and U.S. Pat. No. 7,841,984 which are hereby incorporated by reference.

In one method of the present disclosure, sweat glands tissue in a dermis underlying the epidermis of the skin is transcutaneous contracted with the use of a thermal heating apparatus. Electromagnetic energy is transcutaneous delivered through the epidermis to the underlying dermis.

In one embodiment of the present disclosure, the method comprises the use of an ultrasound HIFU system—in particular, a system with a plurality of independently controlled multiple beam transducer elements that are capable of being focused at the treatment depths below the skin surface.

The treatment begins by applying a HIFU transducer, comprised of an array of transducer elements, to the patient. In one embodiment, the transducer element may comprise a piezoelectric element, a solid coupling element, an air cooling, and a focusing lens. In an exemplary embodiment of the present disclosure five transducer elements span treatment depths including 0.35 to 3.5 cm. The five transducer elements have focal points of 0.5, 0.8, 1.2, 1.9, and 3.0 cm, with correspondingly operate frequencies of 12, 9, 7, 5.5, and 4 MHz, respectively. In a preferred embodiment, the operate frequencies is around 2 Mhz. It should be noted that the transducer may comprise different numbers of transducer elements. In one aspect of the present invention, the HIFU transducer may comprise a plurality of independently movable HIFU transducer elements assembled in a matrix. Accordingly, each individual transducer element may be moved with an “orbital” type motion, much like motion restricted to that of a “ball and socket” joint. This orbital type of motion allows the focal point of ablation to describe a circular region in the tissue, thus applying energy to more tissue than would be delivered if the individual transducers were fixed and immobile. Circles of treated tissue created by this motion may overlap one another or may be sized so as to not overlap. In one aspect of the invention, the transducer elements may be positioned and operated such that, after emitting a series of ultrasonic pulses, a pattern of points of treated (destroyed) tissue may be formed instead of a pattern of circles of treated tissue. A variety of mechanical means well known in the art may be employed to drive and control the motion of the transducer elements.

In a preferred embodiment the used ultrasound HIFU system for reducing sweat gland and for the treatment of hyperhidrosis is described in WO03/070105A1. A preferred system is commercially available under the trade name “Liposonix®” from Solta Medical, Inc., 25881 Industrial Boulevard Hayward, Calif. 94545, USA or any similar devices.

In one preferred embodiment of the disclosure the electromagnetic energy are ultrasound waves, preferably high intense focused ultrasound of 1 to 6 Mhz, preferably 1.5 to 3 Mhz, more preferably around 2 Mhz.

As mentioned above, one example for an apparatus comprising an electromagnetic energy source is the Liposonix® device or any similar devices.

With the methods according to the present disclosure, the severity of the symptoms of focal hyperhidrosis can be reduced. For example, the severity of excessive sweating or other fluid weeping from the skin, erythematous, leathery appearance and texture, and associated pain and discomfort can be reduced.

In a preferred embodiment of the present disclosure, the methods and uses involve non-invasively treating the affected area with one of the mentioned devices, a gel sheet and a transducer, generally penetrating the skin deeply and creating a lesion in the target area.

In an embodiment, the methods and uses involves at least one or two such applications separated by two to six, and preferably four week intervals.

In another embodiment, local anesthetic cream or injection or block may be applied before the treatment. Alternatively, intravenous deep sedation or anesthesia may be used.

In one embodiment of the present disclosure the Liposonix® system was used for the treatment of hyperhidrosis. The Liposonix® system is used in the prior art to apply high intense focused ultrasound to create heat and shear forces, to destroy target structures in the subcutaneous fat, adipocytes, to achieve nonsurgical body sculpting effects. The lesions induce a wound healing effect, macrophages and other cells clear up the cellular debris of disrupted adipocytes, thus leading to volume loss.

In an preferred embodiment, the energy exposure involves treating the diseased tissues with a high intense focused ultrasound device, such as Liposonix® or similar devices, using ultrasound frequencies around 1 to 6 Mhz, preferably 1.5 to 3 Mhz, more preferably around 2 Mhz.

In an embodiment, the ultrasound device is used with a specific transducer and a gel sheet. In preferred embodiments, the device is used for the treatment and then waiting two to six, and most preferably four, weeks after the first treatment, and then repeating this process one or more times if necessary.

Most preferably, one such ultrasound exposure penetrates the diseased tissue and is absorbed by at least some sweat glands. These sweat glands are then disrupted and then resorbed.

The novel use of the devices leads to a treatment which is non-invasive, which can be of very significant benefit to the health care practitioner and the affected patient. The most preferred method is also believed to be very safe and easy and economical to administer without any significant complications for any patient as a result of the preferred type of treatment and procedure.

Further advantages of the high intense focused ultrasound treatment of hyperhidrosis is likely useable to treat conditions of hyperhidrosis and other conditions, that may not qualify for a diagnosis of hyperhidrosis, but exhibit similar symptoms.

In a further embodiment the present disclosure relates to the treatment of hyperhidrosis like focal hyperhidrosis with radiofrequenncy (RF) energy.

In embodiments according to the present disclosure apparatuses and/or systems were used for applying nonablative capacitive radiofrequency energy.

In one embodiment capacitive radiofrequency energy and surface cooling are applied by use of the Thermacool® TC3 or NXT or CPT from Solta Medical, Inc., 25881 Industrial Boulevard Hayward, Calif. 94545, USA or any similar devices.

In a preferred embodiment the radiofrequency energy device is used with an electrode comprising specific treatment tips. The specific treatment tips can be internally and/or externally cooled.

In an embodiment according to the present disclosure, the Thermacool® TC3 or NXT or CPT was used in the treatment.

In one embodiment, the uses and/or the methods comprises radiation exposure involving treating the diseased tissues with a capacitive, nonablative radiofrequency device such as Thermacool TC3 or NXT or CPT with a specific treatment tip with surface cooling.

In an preferred embodiment, after the first treatment and before the next treatment it is preferred to wait two to six, and most preferably four, weeks, and then repeating this process at one or more times if necessary.

In one embodiment, local anesthetic cream or injection or block may be applied before the treatment. Alternatively, intravenous deep sedation or anesthesia may be used.

In embodiments according to the present disclosure the RF energy has a frequency between 6 and 8 Mhz, between 6.5 and 7.5 Mhz, between 6.6 and 6.8 Mhz and 6.78.

In further embodiments according to the present disclosure the RF energy has power output up to 500 W, in particular up to 400 W.

In an advantageous embodiment, radiofrequency energy with a frequency of 6.78 Mhz with a power output up to 400 W is used and the nonablative energy is applied through an internally cooled treatment tip to create a uniformously heated area at a specific penetration depth to denature effectively a large number of sweat glands.

Most preferably, one such radiofrequency exposure penetrates the diseased tissue and is absorbed by at least some sweat glands. These sweat glands are then heated and destroyed.

“Standard thermal gradient” is the thermal content of tissue that is greater on the skin surface.

“Reverse thermal gradient” is, (i) the application of electromagnetic energy to alter the biophysical properties of sweat glands, i.e., reduction, with minimal blistering of the tissue surface, (ii) a gradient in which the tissue surface temperature is cooler than the underlying sweat glands comprising tissue, (iii) conditions in which a standard thermal gradient is reduced or equalized in temperature between the tissue surface and the underlying sweat glands, or (iv) monitoring the heat content (temperature and exposure duration) of the tissue surface to avoid blistering during treatment, regardless of the tissue surface temperature relative to the underlying sweat glands tissue.

“Transcutaneously” means that the delivery device delivers electromagnetic energy directly through the tissue surface.

“Sweat glands” are small tubular structures of the skin that produce sweat including eccrine sweat glands which can be distributed all over the body although their density varies from region to region and apocrine sweat glands. In advantageous embodiments, the eccrine sweat glands are reduced by the methods according to the present disclosure.

In one embodiment a hand wand is used for the treatment of hyperhidrosis according to the present disclosure, wherein the wand comprises: a first controlling device operably controlling an ultrasonic imaging function for providing ultrasonic imaging; a second controlling device operably controlling an ultrasonic treatment function for providing ultrasonic treatment; a movement mechanism configured to direct ultrasonic treatment in a linear sequence of individual thermal lesions; and at least a first and a second removable transducer module, wherein said first and second transducer modules are configured for both ultrasonic imaging and ultrasonic treatment, wherein said first and second transducer modules are configured for interchangeable coupling to the hand wand, wherein said first transducer module is configured to apply ultrasonic therapy to a first layer of tissue, wherein said second transducer module is configured to apply ultrasonic therapy to a second layer of tissue, wherein the second layer of tissue is at a different depth than the first layer of tissue, and wherein said first and second transducer modules are configured to be operably coupled to at least one of said first controlling device, said second controlling device and said movement mechanism. One example for a system that can be used for the treatment of hyperhidrosis is described in WO2009/149390 A1 which is hereby incorporated by reference.

In several embodiments, a hand wand is used for the treatment of hyperhidrosis according to the present disclosure. In accordance with one embodiment, the hand wand comprises a first controlling device, a second controlling device, a movement mechanism, and a transducer module. The first controlling device operably controls an ultrasonic imaging function for providing ultrasonic imaging. The second controlling device operably controls an ultrasonic treatment function for providing ultrasonic treatment. The movement mechanism is configured to direct ultrasonic treatment in a sequence of individual thepnal lesions. The removable transducer module is configured for both ultrasonic imaging and ultrasonic treatment. The removable transducer module is configured for interchangeable coupling to the hand wand. The removable transducer module is configured to be operably coupled to at least one of said first controlling device, said second controlling device and said movement mechanism. The removable transducer module is configured to apply ultrasonic therapy to at a first variable ultrasonic parameter to tissue.

It is to be understood that the foregoing is merely a brief summary of aspects of the invention. Other aspects, advantages, and objects of the invention will become apparent as the specification proceeds. The scope of the present invention is therefore to be determined by reference to the issued claims and not by whether given subject matter meets all objects or advantages set forth herein or solves, or reduces the severity of, all issues or problems in the prior art noted above.

The following examples and methods are offered for illustrative purposes only, and are not intended to limit the scope of the present disclosure in any way.

### EXAMPLES

A series of clinical studies in human subjects have been performed to support the clinical evaluation of the novel uses of the apparatuses and methods according to the present disclosure.

### Example 1

Patient 1

Patient 1 was 41 yrs and, through steps one and two, diagnosed with Hyperhidrosis axillaris. The disease started at the age of 17 yrs and never stopped. Iontopheresis or systemic therapy was not sufficient. Mycosis appeared. Gravimetry showed excessive sweating of 209 mg/min on the right side and 166 mg/min on the left side. The patient also suffered from discomfort in the diseased area,

The patient's hyperhidrosis condition was stable but included the significant problematic symptoms noted above.

The patient's axillae were treated with the Liposonix® device and a gel sheet, applying high intense focused ultrasound to this patient with the appropriate energy levels and appropriate number of pulses and passes, and with a waiting time of four weeks before repeating the treatment.

Immediately after the first treatment, this patient noted improvement in sweating and discomfort. The patient was treated a second time four weeks later. After the second treatment, this patient's hyperhidrosis was eliminated and the patient was satisfied. Gravimetry showed significant reduction to 28 mg/min on the right side and 27 mg/min on the left side.

Histology showed denatured sweat glands.

### Example 2

Patient 2

Patient 2 was 38 yrs and, through steps one and two, diagnosed with Hyperhidrosis palmaris. The disease started at the age of 15 yrs and never stopped. Iontopheresis or Btx were not sufficient. Gravimetry showed excessive sweating of 154 mg/min on the right side and 191 mg/min on the left side. The patient also suffered from erythema and discomfort in the diseased area,

The patient's hyperhidrosis condition was stable but included the significant problematic symptoms noted above.

The patient's palms were anesthetized with a local anesthetic bloc and then treated with high intense focused ultrasound of Liposonix® through a gel sheet, with the appropriate energy levels and appropriate number of pulses and passes, with a waiting time of four weeks before repeating the treatment.

Immediately after the first treatment, this patient noted improvement in sweating and discomfort. Six weeks later, after the second treatment, this patient's hyperhidrosis was eliminated and the patient was satisfied. Gravimetry showed significant reduction to 11 mg/min on the right side and 34 mg/min on the left side.

### Example 3

Patient 3

Patient 3 was 30 yrs and, through steps one and two, diagnosed with Hyperhidrosis plantaris. The disease started at the age of 18 yrs and never stopped. Iontopheresis was not sufficient. Mycosis appeared. Gravimetry showed excessive sweating of 351 mg/min on the right side and 386 mg/min on the left side. The patient also sulfered from discomfort in the diseased area.

The patient's hyperhidrosis condition was stable but included the significant problematic symptoms noted above.

After deep sedation and application of local anesthetic cream, the patient's feet were treated with high intense focused ultrasound of Liposonix® through a gel sheet, with the appropriate energy levels and appropriate number of pulses and passes, with a waiting time of eight weeks before repeating the treatment and another six weeks before the third treatment.

Immediately after the first treatment, this patient noted improvement in sweating and discomfort. After the third treatment, this patient's hyperhidrosis was eliminated and the patient was satisfied. Gravimetry showed significant reduction to 57 mg/min on the right side and 29 mg/min on the left side.

It can thus be seen from the examples above that the disclosed technology technique using high intense focused ultrasound, to greatly reduce and in some cases eliminate the symptoms of hyperhidrosis. In doing so, this high intense focused ultrasound energy heats and disrupts the target structures, such as sweat glands, until they are destroyed, while other structures such as epidermis superficial to the target or other structures deeper to the target are protected.

### Example 4

Patient 4

Patient 4 was 34 yrs and, through steps one and two, diagnosed with Hyperhidrosis axillaris. The disease started at the age of 15 yrs and never stopped. AICI or iontopheresis were not sufficient. Intertrigo appeared. Gravimetry showed excessive sweating of 188 mg/min on the right side and 231 mg/min on the left side. The patient also suffered from discomfort in the diseased area.

The patient's hyperhidrosis condition was stable but included the significant problematic symptoms noted above.

The patient's axillae was treated with a deep treatment tip, applying non ablative capacitive radiofrequency radiation to this patient with the appropriate energy levels and appropriate number of pulses, while cooling the skin prior, during and post the pulse and with a waiting time of four weeks before repeating the treatment.

Immediately after the first treatment, this patient noted improvement in sweating and discomfort. The patient was treated a second time four weeks later. After the second treatment, this patient's hyperhidrosis was eliminated and the patient was satisfied. Gravimetry showed significant reduction to 17 mg/min on the right side and 32 mg/min on the left side.

### Example 5

Patient 5

Patient 5 was 28 yrs and, through steps one and two, diagnosed with Hyperhidrosis palmaris. The disease started at the age of 16 yrs and never stopped. AICI or iontopheresis were not sufficient. Injections with Botox were sufficient for 4 mths. Gravimetry showed excessive sweating of 197 mg/min on the right side and 178 mg/min on the left side. The patient also suffered from erythema and discomfort in the diseased area.

The patient's hyperhidrosis condition was stable but included the significant problematic symptoms noted above.

The patient's palms were anesthetized with a prilocaine-lidocaine-cream and then treated with a deep treatment tip, applying non ablative capacitive radiofrequency radiation to this patient with the appropriate energy levels and appropriate number of pulses, while cooling the skin surface prior, during and post the pulse and with a waiting time of four weeks before repeating the treatment.

Immediately after the first treatment, this patient noted improvement in sweating and discomfort. Six weeks later, after the second treatment, this patient's hyperhidrosis was eliminated and the patient was satisfied. Gravimetry showed significant reduction to 41 mg/min on the right side and 20 mg/min on the left side. Histology showed denatured sweat glands.

### Example 6

Patient 6

Patient 6 was 31 yrs and, through steps one and two, diagnosed with Hyperhidrosis plantaris. The disease started at the age of 14 yrs and never stopped. AICI or iontopheresis were not sufficient. Mycosis appeared. Gravimetry showed excessive sweating of 238 mg/min on the right side and 310 mg/min on the left side. The patient also suffered from discomfort in the diseased area.

The patient's hyperhidrosis condition was stable but included the significant problematic symptoms noted above.

After a local anesthetic block, the patient's feet were treated with a deep treatment tip, applying non ablative capacitive radiofrequency radiation to this patient with the appropriate energy levels and appropriate number of pulses, while cooling the skin prior, during and post the pulse and with a waiting time of four weeks before repeating the treatment.

Immediately after the first treatment, this patient noted improvement in sweating and discomfort. Four weeks after the second treatment, this patient's hyperhidrosis was eliminated and the patient was satisfied. Gravimetry showed significant reduction to 55 mg/min on the right side and 35 mg/min on the left side.

Patient 7

Patient 7 was 42 yrs and, through steps one and two, diagnosed with Hyperhidrosis axillaris. The disease started at the age of 14 yrs and never stopped. Pretreatments were not sufficient. Erythma appeared. Gravimetry showed excessive sweating of 138 mg/min on the right side and 149 mg/min on the left side. The patient also suffered from discomfort in the diseased area.

The patient's hyperhidrosis condition was stable but included the significant problematic symptoms noted above.

After application of a local anesthetic creme, the patient's axilla were treated with a face CPT Thermage treatment tip, applying non ablative capacitive radiofrequency radiation to this patient with the appropriate energy levels and appropriate number of pulses, while cooling the skin prior, during and post the pulse and with a waiting time of four weeks before repeating the treatment.

Immediately after the first treatment, this patient noted improvement in sweating and discomfort. Immediately after the second treatment, this patient's hyperhidrosis was eliminated and the patient was satisfied. Gravimetry showed significant reduction to 20 mg/min on the right side and 20 mg/min on the left side.

Patient 8

Patient 8 was 22 yrs and, through steps one and two, diagnosed with Hyperhidrosis plantaris. The disease started at the age of 12 yrs and never stopped. AICI was not sufficient. Gravimetry showed excessive sweating of 178 mg/min on the right side and 170 mg/min on the left side. The patient also sulfered from discomfort in the diseased area.

The patient's hyperhidrosis condition was stable but included the significant problematic symptoms noted above.

After application of a local anesthetic creme, the patient's axillae were treated with high intense focused ultrasound of Liposonix® through a gel sheet, with the appropriate energy levels and appropriate number of pulses and passes, with a waiting time of four weeks before repeating the treatment.

Immediately after the first treatment this patient's hyperhidrosis was eliminated and the patient was satisfied. Gravimetry showed significant reduction to 30 mg/min on the right side and 20 mg/min on the left side.

Embodiments of the present disclosure pertain to:
Use of electromagnetic energy for reducing sweat glands in the treatment of hyperhidrosis, wherein:
   a) An electromagnetic energy source is provided;
   b) Energy is delivered from the energy source through the skin surface to the sweat glands containing tissue site, wherein the temperature of the skin surface is less than the temperature of the sweat glands containing tissue site; and
   c) the sweat glands are reduced.
wherein the electromagnetic energy source may be an electrode means for delivering thermal energy.
wherein the electrode means may be a radiofrequency (RF) electrode coupled to an RF energy source for nonablative capacitive radiofrequency radiation.
may further comprising a source of electrolytic solution that delivers electrolytic solution to the RF electrode.
wherein RF energy may be transferred from the RF electrodes to the electrolytic solution.
wherein the RF energy may has a frequency between 6 and 8 Mhz.
wherein the RF energy may has a frequency between 6.5 and 7.5 Mhz.
wherein the RF energy may has a frequency between 6.6 and 6.8 Mhz.
wherein the RF energy may has a frequency of 6.78 Mhz.
Wherein the RF energy may has power output up to 500 W.
wherein the RF energy may has power output up to 400 W.
wherein an internally cooled treatment tip may be used.
wherein the electrode means may be an ultrasound emitter coupled to an ultrasound energy source.
wherein the ultrasound emitter may emits high intense focused ultrasound.
wherein the ultrasound may has a frequency of approximately 2 MHz.
wherein the hyperhidrosis may be focal hyperhidrosis.
   Further embodiments of the present disclosure pertain to:

A cosmetic method for the treatment of hyperhidrosis, comprising:

providing an electromagnetic energy source including an electrode means for delivering thermal energy through an interface surface and an external skin surface to an underlying sweat glands containing tissue site; positioning the apparatus interface surface adjacent to the external skin surface; delivering sufficient thermal energy from the electrode means through the external skin surface to sweat glands containing tissue site; and reducing the sweat glands.
wherein the electrode means may be a radiofrequency (RF) electrode coupled to an RF energy source applying nonablative capacitive radiofrequency radiation.
May further comprise a source of electrolytic solution that delivers electrolytic solution to the RF electrode.
wherein RF energy may be transferred from the RF electrodes to the electrolytic solution.
wherein the RF energy may has a frequency between 6 and 8 Mhz.
wherein the RF energy may has a frequency between 6.5 and 7.5 Mhz.
wherein the RF energy may has a frequency between 6.6 and 6.8 Mhz.
wherein the RF energy may has a frequency of 6.78 Mhz.
wherein the RF energy may has power output up to 500 W.
wherein the RF energy may has power output up to 400 W.
wherein an internally cooled treatment tip may be used.
wherein the electrode means may be an ultrasound emitter coupled to an ultrasound energy source.
wherein the ultrasound emitter may emits high intense focused ultrasound.
wherein the ultrasound may has a frequency of at least 2 MHz.
wherein the hyperhidrosis may be focal hyperhidrosis.

1-41. (canceled)

42. A method for reducing sweat glands in a selected sweat glands containing tissue site beneath an epidermis skin surface, comprising:
a) providing a thermal energy source with an energy delivery surface;
b) positioning the energy delivery surface on the epidermis skin surface;
c) creating an uniform reverse thermal gradient through the skin epidermis surface where a temperature of the skin epidermis surface is lower than the selected sweat glands containing tissue site; and
d) delivering energy from the energy source through the skin epidermis surface to the selected sweat glands containing tissue site for a sufficient time to reduce a plurality of sweat glands in the selected sweat glands containing tissue site.

43. (canceled)

44. (canceled)

45. The method according to claim 42, wherein the energy source is a radiofrequency (RF) energy source, and wherein a radiofrequency (RF) electrode is coupled to the RF source for nonablative capacitive radiofrequency radiation.

46. (canceled)

47. (canceled)

48. The method according to claim 45, wherein the RF energy has a frequency between 6 and 8 Mhz, in particular between 6.5 and 7.5 Mhz, in particular between 6.6 and 6.8 Mhz, in particular a frequency of 6.78 Mhz.

49. The method according to claim 45, wherein the RF energy has power output up to 500 W, preferably up to 400 W.

50. The method according to claim 45, wherein an internally cooled treatment tip for applying the RF energy to the sweat glands containing tissue site beneath an epidermis skin surface is used.

51. (canceled)

52. The method of claim 42, wherein the energy source is a high intensity focused ultrasound (HIFU) source.

53. (canceled)

54. (canceled)

55. The method according to claim 52, wherein the ultrasound has a frequency of approximately 2 MHz.

56. The method according to claim 42, wherein the skin surface is cooled with a gel sheet.

57. (canceled)

58. (canceled)

59. The method according to claim 42, wherein the selected sweat glands containing tissue site is heated to a temperature range of 40 to 60 degrees C.

60. The method according to claim 42, wherein the selected sweat glands containing tissue site is heated to a temperature range of 60 to 80 degrees C.
